(19) United States
(12) Patent Application Publication
(10) Pub. No.: US 2016/0264565 A1
(43) Pub. Date: Sep. 15, 2016

(54) CRYSTALLINE DASATINIB PROCESS

(71) Applicant: SHILPA MEDICARE LIMITED, RAICHUR (IN)

(72) Inventors: SRIRAM RAMPALLI, VIZIANAGARAM (IN); PRADEEP POTHANA, VIZIANAGARAM (IN); SURESH GARBAPU, VIZIANAGARAM (IN); PRASHANT PUROHIT, RAICHUR (IN); AKSHAYKANT CHATURVEDI, RAICHUR (IN)

(73) Assignee: SHILPA MEDICARE LIMITED, RAICHUR (IN)

(21) Appl. No.: 15/033,910

(22) PCT Filed: Apr. 24, 2014
(86) PCT No.: PCT/IB2014/060968

§ 371 Date: May 3, 2016

(30) Foreign Application Priority Data
Nov. 8, 2013 (IN) 5038/CHE/2013

(51) Int. Cl. C07D 417/12; C07H 3/02

(52) U.S. Cl. CPC C07D 417/12; C07H 3/02; C07B 2200/13

(57) ABSTRACT

The present invention relates to a process for preparation of crystalline Form-SDI of Dasatinib (I).

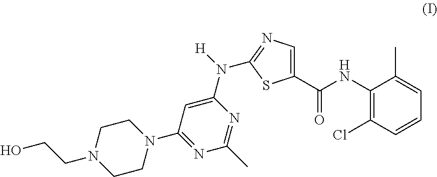 [SMILES: Cc1nc(Nc2ncc(C(=O)Nc3c(C)cccc3Cl)s2)cc(N2CCN(CCO)CC2)n1]

Said crystalline Form-SDI of Dasatinib is characterized by X-ray powder diffraction pattern comprising of at least seven 2θ° peaks selected from the XRPD peak set of 5.8, 11.5, 12.7, 13.2, 17.3, 17.5, 18.1, 20.1, 20.5, 22.1, 25.4, 26.6, 26.8±0.20 2θ°; IR spectrum having at least five absorption peaks selected from about 3390 cm⁻¹, 2923 cm⁻¹, 1621 cm⁻¹, 1615 cm⁻¹, 1537 cm⁻¹, 1316 cm⁻¹, 1061 cm⁻¹, 815 cm⁻¹ and 783 cm⁻¹; and DSC isotherm comprising at least two endothermic peaks ranging between −130° C. to 150° C., 160° C. to 175° C. or 280° C. to 290° C.

The pharmaceutical compositions of the crystalline Form-SDI of Dasatinib or its hydrate thereof may be useful as an anti-cancer agent.

### FIELD OF INVENTION

The present invention relates to a process for preparation of crystalline Form-SDI of Dasatinib (I).

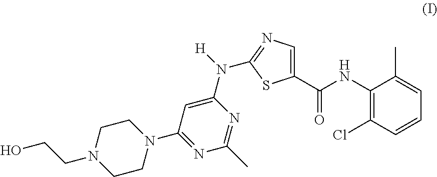 [SMILES: Cc1nc(Nc2ncc(C(=O)Nc3c(C)cccc3Cl)s2)cc(N2CCN(CCO)CC2)n1]

Said crystalline Form-SDI of Dasatinib is characterized by X-ray powder diffraction pattern comprising of at least seven 2θ° peaks selected from the XRPD peak set of 5.8, 11.5, 12.7, 13.2. 17.3, 17.5, 18.1, 20.1, 20.5, 22.1, 25.4, 26.6, 26.8±0.20 2θ°; IR spectrum having at least five absorption peaks selected from about 3390 cm⁻¹, 2923 cm⁻¹, 1621 cm⁻¹, 1615 cm⁻¹, 1537 cm⁻¹, 1316 cm⁻¹, 1061 cm⁻¹, 815 cm⁻¹ and 783 cm⁻¹, and DSC isotherm comprising at least two endothermic peaks ranging between −130° C. to 150° C., 160° C. to 175° C. or 280° C. to 290° C.

The pharmaceutical compositions of the crystalline Form-SDI of Dasatinib or its hydrate thereof may be useful as an anti-cancer agent.

### BACKGROUND OF THE INVENTION

Dasatinib is chemically described as N-(2-chloro-6-methylphenyl)-2-[[6-[4-(2-hydroxyethyl)-1-piperazinyl]-2-methyl-4-pyrimidinyl]amino]-5-thiazolecarboxamide and is represented by Formula (I).

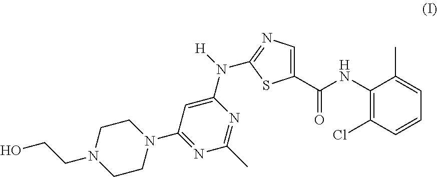 [SMILES: Cc1nc(Nc2ncc(C(=O)Nc3c(C)cccc3Cl)s2)cc(N2CCN(CCO)CC2)n1]

The monohydrate form of Dasatinib is a kinase inhibitor and has been approved by USFDA as SPRYCEL™ for the treatment of chronic phase Philadelphia chromosome-positive (Ph+) Chronic Myeloid Leukemia (CML), in newly diagnosed adult patients or patients having resistance or intolerance to prior therapy like Imatinib. SPRYCEL™ is also indicated for the treatment of adult patients with Philadelphia chromosome-positive acute lymphoblastic leukemia (Ph+) ALL) with resistance or intolerance to prior therapy.

Das et al in U.S. Pat. No. 6,596,746 B1 provided the first disclosure of the compound Dasatinib along with the process for preparation thereof Further to this Lajeunesse et al. in U.S. Pat. No. 7,491,725 B2 provided the crystalline monohydrate, crystalline butanol solvate, crystalline ethanol solvate and neat forms of Dasatinib.

Chidambaram et al in WO2007035874 A1 disclosed various pharmaceutically acceptable salt forms of Dasatinib. The pharmaceutically acceptable salts disclosed in WO2007035874 A1 are for example, fumaric acid, hydrobromic acid, maleic acid, methanesulfonic acid, phosphoric acid, salicylic acid, sulfuric acid, tartaric acid, or p-toluenesulfonic acid.

Amongst other disclosures, Vraspir et al in WO2010062715 A2 disclosed isosorbide dimethyl ether solvate, N,N′-dimethylethylene urea solvate and N,N′-dimethyl-N,N′-propylene urea solvate of Dasatinib. Parthasaradhi et al in WO 2010067374 A2 disclosed crystalline solvates of Dasatinib with DMF, DMSO, toluene, isopropyl acetate and processes for their preparation. Also, Sudershan et al in WO2012014149 A1 disclosed N-Methylformamide solvate of Dasatinib.

Several crystalline forms of Dasatinib are described in the literature; these are designated as H1-7. BU-2, E2-1, N-6, T1H1-7. and T1E2-1. Crystalline dasatinib monohydrate (H1-7) and butanol solvate (BU-2)) along with the processes for their preparation are described in WO 2005077945. Additionally US 2006/0004067, also describe two ethanol solvates (E2-1; T1E2-1) and two anhydrous forms (N-6; T1H1-7).

WO 2009053854 discloses various Dasatinib solvates including their crystalline form, amorphous form and anhydrous form. U.S. Pat. No. 7,973,045 further discloses the anhydrous form of Dasatinib and process for preparation thereof. The anhydrous form disclosed therein has typical characteristic XRD peaks at about 7.2, 11.9, 14.4. 16.5, 17.3, 19.1, 20.8, 22.4, 23.8, 25.3 and 29.1 on the 2-theta value. Further U.S. Pat. No. 8,067,423 B2 discloses crystalline forms of isopropyl alcohol solvate of Dasatinib, along with many other solid state forms of Dasatinib.

WO 2010062715 discloses isosorbide dimethyl ether solvate, N,N′-dimethylethylene urea solvate and N,N′-dimethyl-N,N′-propylene urea solvate of Dasatinib.

WO 2010067374 discloses novel crystalline form I, solvates of DMF, DMSO, toluene, isopropyl acetate and processes for their preparation.

WO 2010139979 discloses MDC solvate of Dasatinib, process of its preparation and use in the manufacture of pure Dasatinib.

WO 2010139980 discloses a process for the preparation of crystalline Dasatinib monohydrate.

Existence of (pseudo)polymorphism is known to be unique phenomenon in solid materials, wherein existence of different physical forms including shape, size, and arrangement of molecules in the physical state or polymorphs of same compound are known in the nature. A single compound, or a salt complex, may give rise to a variety of solids having distinct physical properties, which often results in substantial differences in bioavailability, stability, and other differences between production lots of formulated pharmaceutical products. Due to this reason, since (pseudo)polymorphic forms can vary in their chemical and physical properties, regulatory authorities often require that efforts be made to identify all forms, e.g., hydrate or anhydrate, crystalline or amorphous, solvated or un-solvated forms, etc. of the drug substances.

Some of the new polymorphic forms may turn out to be more efficacious than the other already reported forms. It has generally been observed that some forms of a compound have improved physical and chemical properties without affecting the pharmacological action of the drug and hence provide an opportunity to improve the drug performance characteristics of such product. However, the existence, and possible number, of (pseudo)polymorphic forms for a given compound cannot be predicted. In addition, there are no “standard” procedures that can be used to prepare different (pseudo)polymorphic forms of a substance.

Dasatinib being an important drug in the treatment of cancer, there still appears to be a need for new forms of Dasatinib having further improved physical and/or chemical properties. Hence it was thought worthwhile by the inventors of the present application to explore novel forms of Dasatinib, which may further improve the characteristics of drug Dasatinib. Inventors of the present application here in this application report crystalline Form-SDI of Dasatinib or its hydrate thereof. Crystalline Form-SDI of Dasatinib is sufficiently stable and pharmaceutically acceptable. This stable form offers various advantages in terms of storage, shelf life and favorable impurity profile.

Present invention also provides a process for preparation of crystalline Form-SDI of Dasatinib, which is industrially amenable and commercially viable. Crystalline Form-SDI of Dasatinib can be a valuable input for the preparation of various pharmaceutically acceptable pure salt forms of Dasatinib without any requirement for repeated purification processes, thus giving both economic and operational advantage.

### SUMMARY OF THE INVENTION

Particular aspects of the present specification relate to the novel crystalline Form-SDI of Dasatinib (I) or its hydrate thereof and processes for its preparation. Further, the invention of this application also relates to pharmaceutical compositions comprising of crystalline Form-SDI of Dasatinib (I) or its hydrate thereof, which may be useful in the treatment of various cancerous disorders.

In an aspect of the present application, it relates to a process for preparation of crystalline Form-SDI of Dasatinib (I).

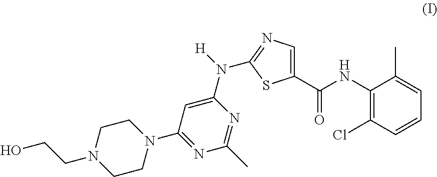 [SMILES: Cc1nc(Nc2ncc(C(=O)Nc3c(C)cccc3Cl)s2)cc(N2CCN(CCO)CC2)n1]

comprising the steps of:

a) Reacting N-(2-chloro-6-methylphenyl)-2-[6-chloro-2-methyl-4-pyrimidinyl) amino]-5-thiazole carboxamide (II) with 1-(3-Hydroxy)ethylpiperazine (III) in solvent 3-methylbutan-1-ol in absence of base;

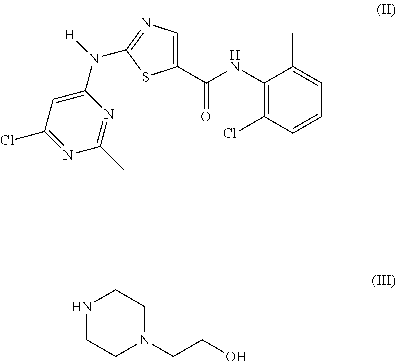 [SMILES: Cc1nc(Cl)cc(Nc2ncc(C(=O)Nc3c(C)cccc3Cl)s2)n1 ; OCCN1CCNCC1]

b) Raising the temperature of reaction mixture to a temperature above 100° C.;

c) Cooling the reaction mixture to room temperature;

d) Filtering the reaction mass and washing it with 3-methylbutan-1-ol;

e) Optionally treating the mass obtained from step d) with X—CH₂—CH₂—OH in presence of an organic base and 3-methylbutan-1-ol; wherein X is halogen;

f) Isolating the crystalline Form-SDI of Dasatinib (I).

Another aspect of the present invention provides crystalline Form-SDI of Dasatinib, which is characterized by X-ray powder diffraction pattern comprising of at least seven 2θ° peaks selected from the XRPD peak set of 5.8, 11.5, 12.7, 13.2, 17.3, 17.5, 18.1, 20.1, 20.5, 22.1, 25.4, 26.6, 26.8±0.20 2θ°; IR spectrum having at least five absorption peaks selected from about 3390 cm⁻¹, 2923 cm⁻¹, 1621 cm⁻¹, 1615 cm⁻¹, 1537 cm⁻¹, 1316 cm⁻¹, 1061 cm⁻¹, 815 cm⁻¹ and 783 cm⁻¹; and DSC isotherm comprising at least two endothermic peaks ranging between −130° C. to 150° C., 160° C. to 175° C. or 280° C. to 290° C.

In another aspect of the present application, crystalline Form-SDI as obtained by the process of the present invention, can be further reacted to obtain pharmaceutically acceptable salts of Dasatinib selected from Dasatinib Glucuronate (A) or Dasatinib hydrochloride (B).

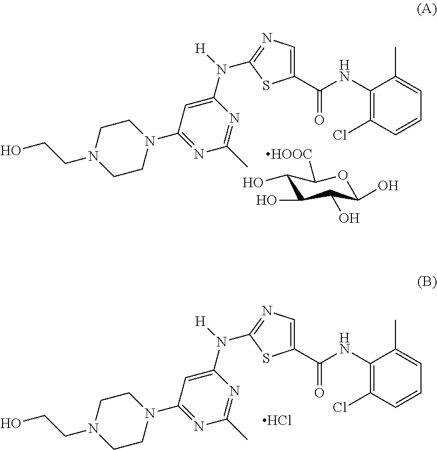

Further aspect of the present invention relates to a composition comprising crystalline Form-SDI of Dasatinib or a hydrate thereof together with at least one or more pharmaceutically acceptable excipient.

Further particular aspects of the present invention are detailed in the description of the invention, wherever appropriate.

### BRIEF DESCRIPTION OF THE DRAWINGS

FIG. 1 is an example of X-ray powder diffraction (“XRPD”) pattern of crystalline Form-SDI of Dasatinib.

FIG. 2 is an example of IR spectral pattern of crystalline Form-SDI of Dasatinib.

FIG. 3 is an example of Differential Scanning calorimetry (“DSC”) curve of crystalline Form-SDI of Dasatinib.

### ABBREVIATIONS

| | |
|---|---|
| CML | Chronic Myeloid Leukemia |
| DIPEA | DiIsoPropyl Ethyl Amine |
| DMF | DiMethyl Formamide |
| DMSO | DiMethyl SulfOxide |
| DSC | Differential Scanning Calorimetry |
| HPLC | High-Performance Liquid Chromatography |
| IR | InfraRed |
| ¹H NMR | Proton Nuclear Magnetic Resonance |
| MDC | Methylene DiChloride |
| MTBE | Methyl Tert-Butyl Ether |
| RBF | Round-Bottom Flask |
| RT | Room Temperature |
| THF | TetraHydroFuran |
| XRPD | X-Ray Powder Diffraction Pattern |

### DETAILED DESCRIPTION

As set forth herein, embodiments of the present invention relate to the novel crystalline Form-SDI of Dasatinib (I) or its hydrate thereof and processes for its preparation.

In one embodiment of the present application, it provides a process for preparation of crystalline Form-SDI of Dasatinib (I).

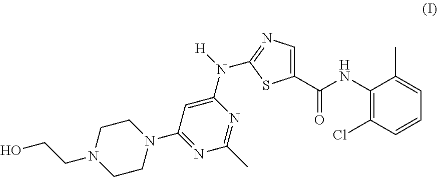 [SMILES: Cc1nc(Nc2ncc(C(=O)Nc3c(C)cccc3Cl)s2)cc(N2CCN(CCO)CC2)n1]

comprising the steps of:

a) Reacting N-(2-chloro-6-methylphenyl)-2-[6-chloro-2-methyl-4-pyrimidinyl) amino]-5-thiazole carboxamide (II) with 1-(3-Hydroxy)ethylpiperazine (III) in solvent 3-methylbutan-1-ol in absence of base;

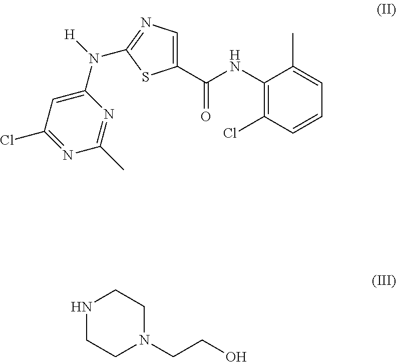 [SMILES: Cc1nc(Cl)cc(Nc2ncc(C(=O)Nc3c(C)cccc3Cl)s2)n1 ; OCCN1CCNCC1]

b) Raising the temperature of reaction mixture to a temperature above 100° C.;

c) Cooling the reaction mixture to room temperature;

d) Filtering the reaction mass and washing it with 3-methylbutan-1-ol;

e) Optionally treating the mass obtained from step d) with X—CH₂—CH₂—OH in presence of an organic base and 3-methylbutan-1-ol; wherein X is halogen;

f) Isolating the crystalline Form-SDI of Dasatinib (I).

The individual steps of the process according to the present invention for preparing crystalline Form-SDI of Dasatinib are detailed separately herein below.

Step a) comprises reacting N-(2-chloro-6-methylphenyl)-2-[6-chloro-2-methyl-4-pyrimidinyl) amino]-5-thiazole carboxamide (II) with 1-(3-Hydroxy)ethylpiperazine (III) in solvent 3-methylbutan-1-ol in absence of base;

N-(2-chloro-6-methylphenyl)-2-[6-chloro-2-methyl-4-pyrimidinyl) amino]-5-thiazole carboxamide (II) and 1-(3-Hydroxy)ethylpiperazine (III) are reacted in solvent 3-methylbutan-1-ol. The reaction is carried out by stirring for 10-30 mins at an ambient temperature of 25-30° C. The amount of solvent used in this reaction ranges from 20-30 times in volume (mL) as compared to weight (g) of Compound (II). Studies carried out by the inventors of the present application showed that use of a base in this step can lead an un-favorable impurity profile of the final product, wherein purification of the end product can lead to a substantial yield loss and some of the impurities can't even be removed by conventional process.

Novel inventive merit of the present invention provides that condensation of compounds of Formula (II) and Formula (III) to be carried out in absence of any base, thus providing the final product in good yield and with a favorable safety profile.

Step b) comprises raising the temperature of reaction mixture to a temperature above 100° C.;

The reaction mixture obtained from step a) is heated to a temperature above 100° C. In a preferred embodiment the reaction mixture is heated to a temperature of 130-140° C. At the raised temperature of above 100° C., the reaction mixture is subjected to stirring for time duration ranging from 2-20 hrs, depending upon the progress of the reaction, as is checked intermittently at regular intervals.

Step c) comprises cooling the reaction mixture to room temperature;

On completion of the reaction as mentioned in step b), the reaction mixture is slowly cooled to room temperature (˜25-30° C.). Drastic cooling of the reaction mixture shall be avoided so as to avert formation of unwanted impurities. Rate of cooling may be required to be controlled artificially depending upon the environmental conditions. In a preferred embodiment, the reaction mixture was cooled from 135° C. to 25° C. in 2 hrs. When the reaction mixture attains the room temperature, it is subjected to further stirring for time duration ranging from 1-7 hrs.

Step d) comprises filtering the reaction mass and washing it with ethylbutan-1-ol;

The reaction mass obtained from step c) is filtered and washed with 3-methylbutan-1-ol, Filtration may be done by any conventional method known to the person having ordinary skill in the art. Amount of 3-methylbutan-1-ol used in this step ranges from 1-5 times in volume (mL) as compared to weight (g) of Compound (II) taken initially in step a).

Step e) comprises optionally treating the mass obtained from step d) with X—CH2—CH2—OH in presence of an organic base and 3-methylbutan-1-ol; wherein X is halogen;

The reaction mass obtained from step d), is optionally treated with compound of formula X—CH2—CH2—OH in presence of an organic base and 3-methylbutan-1-ol. X in compound of formula X—CH2—CH2—OH, is halogen. In one of the preferred embodiment, X—CH2-CH2—OH is represented by 2-bromoethanol.

Organic base used in this step is an amine or nitrogen-containing heterocyclic compound selected from diisopropyl ethyl amine, pyridine, methyl amine, trimethylamine or triethyl amine. In one of the preferred embodiment, organic base used is diisopropyl ethyl amine (DIPEA).

Amount of 3-methylbutan-1-ol used in this reaction ranges from 10-20 times in volume (mL) as compared to weight (g) of crude reaction mass obtained from step d). The reaction is initially carried out at RT (˜25-30° C.) and then slowly the reaction temperature is raised to a temperature above 60° C., preferably about 80-85° C. At this raised temperature, the reaction mixture is stirred for time ranging from 5-15 hrs. After the completion of the reaction as confirmed by HPLC, the reaction mixture is allowed to slowly cool down to 25-30° C., wherein stirring was performed for time duration ranging from 30 mins to 2 hrs.

Step f) comprises isolating the crystalline Form-SDI of Dasatinib (I).

The reaction mass obtained from step e) is filtered and given washing with 3-methylbutan-1-ol. The wet compound is unloaded and subjected to air drying for ˜30 mins at RT. The partially dried material is subjected to further drying under reduced pressure conditions at temperature of 60-65° C. Drying under reduced pressure conditions may be carried out for time duration ranging from 5-20 hrs. When no further weight loss is observed on drying, the dryer temperature is allowed to cool down to RT and crystalline material is isolated as Form-SDI of Dasatinib.

Process of isolating crystalline Form-SDI of Dasatinib may comprise processes but not limited to conventional processes including scrapping, if required filtering from slurry and optional further drying, which may be carried out at room temperature for the suitable durations to retain the characteristics of crystalline Form-SDI of Dasatinib.

The process of the present invention is advantageous in being commercially viable and industrially feasible as the crystalline Form-SDI of Dasatinib is directly obtained from the reaction without any requirement for initial isolation of Dasatinib base and its further conversion to the required polymorphic form.

The process related impurities, including unreacted intermediates, side products, degradation products and other medium dependent impurities, that appear in the impurity profile of the Dasatinib may be substantially removed by the process of the present invention resulting in the formation of pure Form-SDI of Dasatinib in high yield. In view of maintaining the equilibrium to the impurity profile compliance, the process may require in-process quality checks to avoid unnecessary prolongation/repetitions of the same process steps. Substantially pure Form-SDI of Dasatinib obtained according to the process of the present invention results in the final API purity by HPLC of more than 99% w/w.

The crystalline Form-SDI of Dasatinib obtained by the process of the present invention can be further used for the preparation of substantially pure pharmaceutically acceptable salts of Dasatinib. In a preferred embodiment of the present invention, crystalline Form-SDI of Dasatinib is used as starting material for the preparation of Dasatinib Glucuronate, according to the process covered in our co-pending application IN/3272/CHE/2013. Other non-limiting examples of salts of Dasatinib that can be prepared from crystalline Form-SDI of Dasatinib include Dasatinib hydrochloride. Conversion of crystalline Form-SDI of Dasatinib to other salt forms of Dasatinib can be achieved by the person skilled in the art according to any process available in the prior art. In a preferred embodiment, preparation of the pharmaceutically acceptable salt of Dasatinib shall involve the treatment of the reaction mass obtained as end product, with an organic solvent characterized by boiling point of less than 70° C. Organic solvent characterized by boiling point of less than 70° C. may be selected from ether solvents like Diisopropyl ether, Tetrahydrofuran (THF) and MTBE or a mixture thereof. Use of crystalline Form-SDI as starting material is advantageous for preparation of pharmaceutically acceptable salts of Dasatinib as the required salt forms are obtained in the substantially pure form without any requirement for repeated purification processes, thus giving both economic and operational advantage.

Crystalline Form-SDI of Dasatinib is found to be a very stable crystal lattice which is adequately stable to handle and store for longer time without any significant or measurable change in its morphology and physicochemical characteristics. Crystalline Form-SDI of Dasatinib retains its stoichiometry even on drying for more than 20 hrs at 60-65° C. This stable form thus, offers various advantages in terms of storage, shelf life and favorable impurity profile.

Crystalline Form-SDI of Dasatinib is characterized by X-ray powder diffraction pattern comprising of at least seven 20° peaks selected from the XRPD peak set of 5.8, 11.5, 12.7, 13.2, 17.3, 17.5, 18.1, 20.1, 20.5, 22.1, 25.4, 26.6, 26.8±0.20 2θ°.

In one of the embodiments of the present application, Crystalline Form-SDI of Dasatinib according to the present invention is, characterized by an IR absorption spectrum having at least five absorption peaks selected from about 3390 cm⁻¹, 2923 cm⁻¹, 1621 cm⁻¹, 1615 cm⁻¹, 1537 cm⁻¹, 1316 cm⁻¹, 1061 cm⁻¹, 815 cm⁻¹ and 783 cm⁻¹.

In a further embodiment of the present application, the crystalline Form-SDI of Dasatinib produced by the process of the present invention is characterized by—
i. X-ray powder diffraction pattern comprising of at least seven 2θ° peaks selected from the XRPD peak set of 5.8, 11.5, 12.7, 13.2, 17.3, 17.5, 18.1, 20.1, 20.5, 22.1, 25.4, 26.6, 26.8±0.20 2θ° and/or a doublet diffraction angle peak at 23.6 and 23.8±0.20 2θ°.
ii. DSC isotherm comprising at least two endothermic peaks ranging between −130° C. to 150° C., 160° C. to 175° C. or 280° C. to 290° C.
iii. IR absorption spectrum having at least five absorption peaks selected from about 3390 cm⁻¹, 2923 cm⁻¹, 1621 cm⁻¹, 1615 cm⁻¹, 1537 cm⁻¹, 1316 cm⁻¹, 1061 cm⁻¹, 815 cm⁻¹ and 783 cm⁻¹.

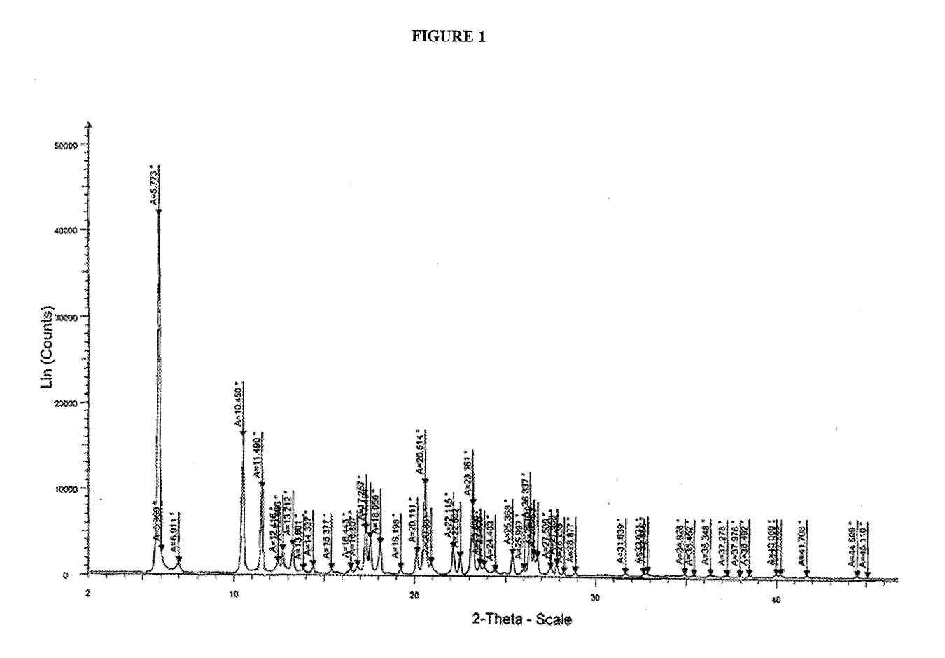

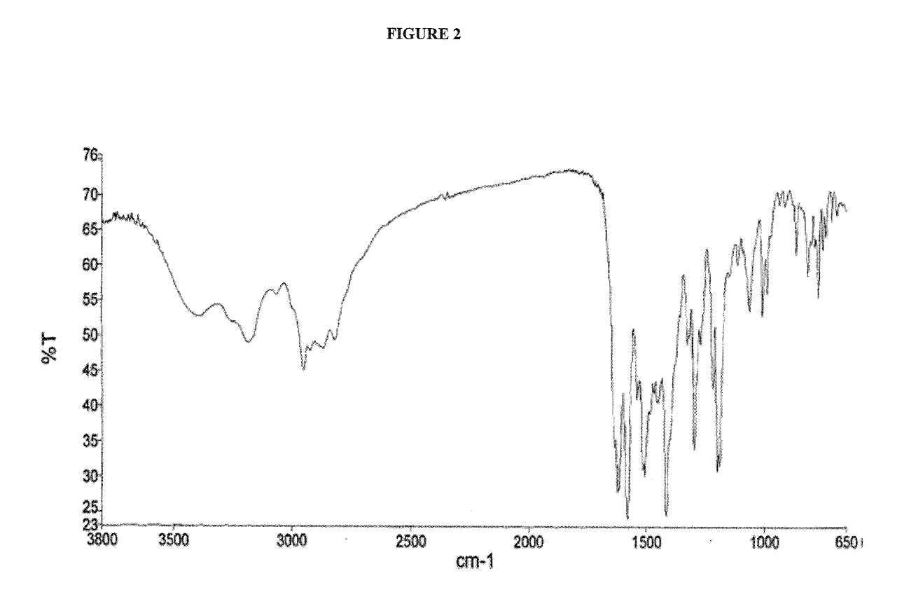

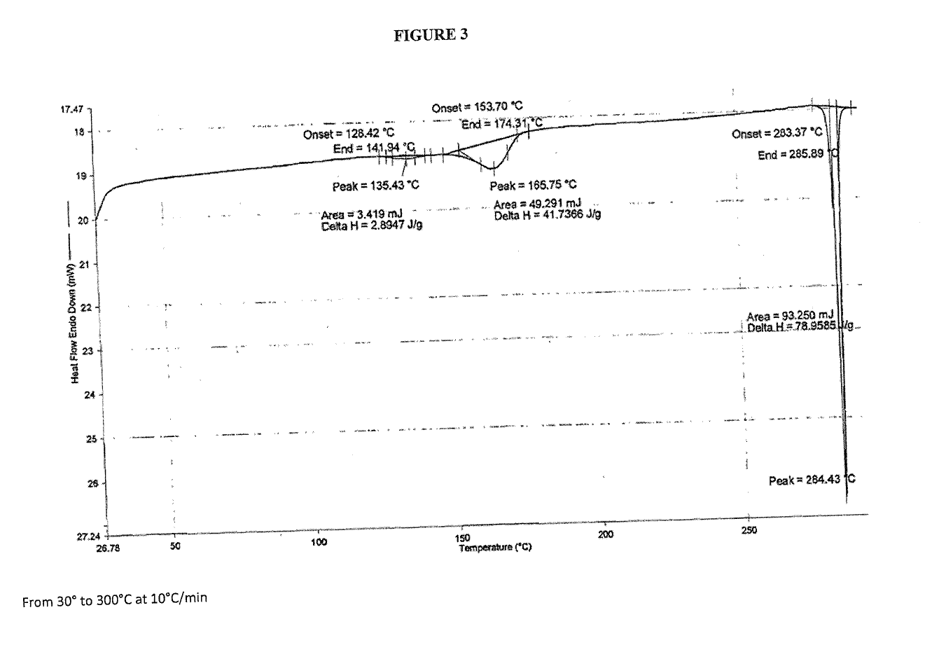

In another embodiment of the present application, substantially pure crystalline Form-SDI of Dasatinib exhibits an X-ray powder diffraction pattern as shown in FIG. 1, IR absorption spectrum as shown in FIG. 2 and DSC isothermal pattern as shown in FIG. 3. In a further embodiment of this application the crystalline Form-SDI of Dasatinib is characterized by X-ray powder diffraction pattern, wherein the diffraction angle peak at 25.4±0.20 2θ° is un-split. The characteristic 2θ° peaks and their d spacing values, for the new crystalline Form-SDI are tabulated in the Table-1.

TABLE 1
Characteristic XRPD Peaks of Crystalline Form-SDI

| | Angle | d Spacing Value |
|---|---|---|
| S. No. | (2θ°) ± 0.20 | (A°) |
| 1. | 5.77 | 15.296 |
| 2. | 11.49 | 7.694 |
| 3. | 12.66 | 6.983 |
| 4. | 13.21 | 6.695 |
| 5. | 17.25 | 5.134 |
| 6. | 17.49 | 5.065 |
| 7. | 18.05 | 4.908 |
| 8. | 20.11 | 4.411 |
| 9. | 20.51 | 4.325 |
| 10. | 22.11 | 4.016 |
| 11. | 23.59 | 3.767 |
| 12. | 23.80 | 3.734 |
| 13. | 25.36 | 3.508 |
| 14. | 26.54 | 3.354 |
| 15. | 26.75 | 3.329 |

Minor variations in the observed 2θ° angles values may be expected based on the analyst, the specific XRPD diffractometer employed and the sample preparation technique. Further possible variations may also be expected for the relative peak intensities, which may be largely affected by the non-uniformity of the particle size of the sample. The 2 theta diffraction angles and corresponding d-spacing values account for positions of various peaks in the X-ray powder diffraction pattern. D-spacing values are calculated with observed 2 theta angles and copper K α wavelength using the Bragg equation well known to those of having skill in the art of XRPD diffractometry science.

In view of possibility of marginal error in the assigning 2 theta angles and d-spacing, the preferred method of comparing X-ray powder diffraction patterns in order to identify a particular crystalline form is to overlay the X-ray powder diffraction pattern of the unknown form over the X-ray powder diffraction pattern of a known form. For example, one skilled in the art can overlay an X-ray powder diffraction pattern of an unidentified crystalline form of Dasatinib over FIG. 1 and readily determine whether the X-ray diffraction pattern of the unidentified form is substantially the same or different w.r.t. the X-ray powder diffraction pattern of the crystalline form SDI of this invention.

The new stable crystalline Form-SDI is characterized by 3-methylbutan-1-ol content in range of 10-16% w/w. Form-SDI has been found to be quite stable and easy to handle and store for longer time without any measurable change in its morphology and physicochemical characteristics, while retaining its properties within the defined limits. Crystalline Form-SDI retains its stoichiometry w.r.t. 3-methylbutan-1-ol content even on drying for more than 20 hrs at 60-65° C. This offers advantages for large scale manufacturing in terms of handling, storage, shelf life and favorable impurity profile.

The crystalline Form-SDI described herein may be characterized by X-ray powder diffraction pattern (XRPD) and Thermal techniques such as differential scanning calorimetry (DSC) analysis. The samples of crystalline Form-SDI of Dasatinib were analyzed by XRPD on a Bruker AXS D8 Advance Diffractometer using X-ray source—Cu Kα radiation using the wavelength 1.5418 Å and lynx Eye detector. DSC was done on a Perkin Elmer Pyris 7.0 instrument. Illustrative examples of analytical data for crystalline Form-SDI of Dasatinib obtained in the Examples are set forth in the FIGS. 1-3.

In a further embodiment according to the specification, the invention also relates to a composition containing crystalline Form-SDI in which at least 95% by total weight of Dasatinib in the composition is in the form of the crystalline Form-SDI. In yet another embodiment of the invention, the composition may be substantially free of any other known forms of Dasatinib.

The crystalline Form-S Dl obtained by the process of the present application may be formulated as solid compositions for oral administration in the form of capsules, tablets, pills, powders or granules. In these compositions, the active product is mixed with one or more pharmaceutically acceptable excipients. The drug substance can be formulated as liquid compositions for oral administration including solutions, suspensions, syrups, elixirs and emulsions, containing solvents or vehicles such as water, sorbitol, glycerin, propylene glycol or liquid paraffin.

In one embodiment of the present invention, it also includes premix comprising one or more pharmaceutically acceptable excipients in the range of 1 to 50% w/w with crystalline Form-SDI, while retaining the nature of the premix.

The compositions for parenteral administration can be suspensions, emulsions or aqueous or non-aqueous sterile solutions. As a solvent or vehicle, propylene glycol, polyethylene glycol, vegetable oils, especially olive oil, and injectable organic esters, e.g. ethyl oleate, may be employed. These compositions can contain adjuvants, especially wetting, emulsifying and dispersing agents. The sterilization may be carried out in several ways. e.g. using a bacteriological filter, by incorporating sterilizing agents in the composition, by irradiation or by heating. They may be prepared in the form of sterile compositions, which can be dissolved at the time of use in sterile water or any other sterile injectable medium.

Pharmaceutically acceptable excipients used in the compositions comprising crystalline Form-SDI of the present application include, but are but not limited to diluents such as starch, pregelatinized starch, lactose, powdered cellulose, microcrystalline cellulose, dicalcium phosphate, tricalcium phosphate, mannitol, sorbitol, sugar and the like; binders such as acacia, guar gum, tragacanth, gelatin, pre-gelatinized starch and the like; disintegrants such as starch, sodium starch glycolate, pregelatinized starch, Croscarmellose sodium, colloidal silicon dioxide and the like; lubricants such as stearic acid, magnesium stearate, zinc stearate and the like; glidants such as colloidal silicon dioxide and the like; solubility or wetting enhancers such as anionic or cationic or neutral surfactants, waxes and the like. Other pharmaceutically acceptable excipients that are of use include but not limited to film formers, plasticizers, colorants, flavoring agents, sweeteners, viscosity enhancers, preservatives, antioxidants and the like.

Pharmaceutically acceptable excipients used in the compositions of crystalline Form-SDI of the present application may also comprise to include the pharmaceutically acceptable carrier used for the preparation of solid dispersion, wherever utilized in the desired dosage form preparation.

Certain specific aspects and embodiments of the present application will he explained in more detail with reference to the following examples, which are provided by way of illustration only and should not be construed as limiting the scope of the invention in any manner.

### EXAMPLE

### Example—01

### Process for Preparation of Crystalline ‘Form-SDI’ of Dasatinib

62.5 mL of 3-methylbutan-1-ol was charged into 4 necked RBF at ˜25° C. 2.5 g of N-(2-chloro-6-methylphenyl)-2-[6-chloro-2-methyl-4-pyrimidinyl) amino]-5-thiazole carboxamide and 4.12 g of 1-(3-Hydroxy)ethylpiperazine were added to the reaction mixture. The reaction mass was stirred for ˜15 mins and then the temperature of reaction mass was raised to 135° C. After stirring the heated reaction mass (along with continuous reaction monitoring), the reaction mass was slowly cooled to 25° C., in 2 h. The cooled reaction mass was then stirred for 5 h, filtered and washed with 5.0 mL 3-methylbutan-1-ol. The material obtained after washing was suck dried for 15 min.

The partially wet material obtained above was charged into a RBF and 48.75 mL 3-methylbutan-1-ol, 73 mg Diisopropylethylamine (DIPEA) and 70 mg 2-bromo ethanol were added to the reaction mixture. The reaction mixture was then heated to ˜80° C., wherein stirring was performed for 12 h. After stirring, the reaction mass was allowed to slowly cool down to ˜25° C. wherein it was again stirred for 1 h. Then the reaction mass was filtered and washed with 6.5 mL of 3-methylbutan-1-ol. The wet material obtained was unloaded, air dried for 30 min and then vacuum dried for 10 h at 65° C. The material was then allowed to cool down to 25° C. and unloaded to obtain 3.0 g crystalline Form-SDI of Dasatinib having XRPD pattern according to FIG. 1, IR spectrum according to FIG. 2 and DSC pattern according to FIG. 3.

Yield: 83% % purity (By HPLC): 99.42%

¹H NMR (400 MHz, DMSO) δ 11.39 (s, 1H), 9.8 (s, 1H), 8.22 (s, 1H), 7.4 (dd, 2H), 7.2 (m, 1H), 6.04 (s, 1H), 4.4 (t, 1H), 3.4 (m, 6H), 2.44 (m, 3H), 2.4 (m, 3H), 2.41 (s, 3H), 2.2 (s, 3H), 1.6 (sep, 1H), 1.3 (q, 2H), 0.85 (d, 6H)

### Example—02

### Process for Preparation of Crystalline ‘Form-SDI’ of Dasatinib

63.0 mL of 3-methylbutan-1-ol was charged into 4 necked RBF at ˜30° C. 2.5 g of N-(2-chloro-6-methylphenyl)-2-[6-chloro-2-methyl-4-pyrimidinyl) amino]-5-thiazole carboxamide and 4.12 g of 1-(3-Hydroxy)ethylpiperazine were added to the reaction mixture. The reaction mass was stirred for ˜10 mins and then the temperature of reaction mass was raised to 140° C. After stirring the heated reaction mass (along with continuous reaction monitoring), the reaction mass was slowly cooled to 30° C. in 2 h. The cooled reaction mass was then stirred for 4 h, filtered and washed with 6.0 mL 3-methylbutan-1-ol. The material obtained after washing was suck dried for 10 min.

The partially wet material obtained above was charged into a RBF and 48.75 mL 3-methylbutan-1-ol, 73 mg Diisopropylethylamine (DIPEA) and 70 mg 2-bromo ethanol were added to the reaction mixture. The reaction mixture was then heated to ˜85° C., wherein stirring was performed for 10 h. After stirring, the reaction mass was allowed to slowly cool down to ˜30° C., wherein it was again stirred for 1 h. Then the reaction mass was filtered and washed with 6.5 mL of 3-methylbutan-1-ol. The wet material obtained was unloaded, air dried for 30 min and then vacuum dried for 12 h at 60° C. The material was then allowed to cool down to 30° C. and unloaded to obtain 3.1 g crystalline Form-SDI of Dasatinib having XRPD pattern similar to FIG. 1, IR spectrum similar to FIG. 2 and DSC pattern similar to FIG. 3.

Yield: 87% % purity (By HPLC): 99.70%

### Example—03

### Process for Preparation of Dasatinib Glucuronate (A) by Using Crystalline ‘Form-SDI’ of Dasatinib

10 mL methanol was charged into a 100 mL round bottomed flask at 25-30° C. and 1.0 g crystalline ‘Form-SDI’ of Dasatinib and 0.35 g Glucuronic acid was added to it. The reaction mixture was stirred for 15 mins, followed by heating to a temperature of ˜65° C. Further stirring of the reaction mixture was performed for 30 mins maintaining the temperature of ˜65° C. Then the reaction mixture was allowed to cool down to a temperature up to ˜25° C.

The reaction mixture was subjected to distillation under vacuum at a temperature of ˜50° C. till approximately 1/10 of initial volume of reaction mixture was left. Then 5.0 mL acetone was added to the reaction mixture. Again the reaction mixture was subjected to distillation under vacuum at temperature of ˜50° C. till approximately 1/10 of initial volume of reaction mixture was left. At the same raised temperature of ˜50° C., 10.0 mL of acetone was added to the reaction mixture and the reaction mixture was allowed to cool to ˜25° C. The obtained reaction mixture was stirred for about 1 h at this temperature. The solid obtained was filtered and washed with 2.0 mL chilled acetone.

The obtained material was dried at ˜60° C. for ˜8 h under vacuum. The dried material was added to 20 mL Methyl t-Butyl Ether (MTBE) and heated for ˜30 mins at temperature of ˜55° C. The solid material obtained was filtered and given washing with 2 mL MTBE.

The reaction mass was then suck dried and the wet material obtained was unloaded. The wet material was further dried under vacuum at a temperature of ˜60° C. for 10 hrs, to obtain 1.05 g Dasatinib Glucuronate.

Yield: 87% % purity (By HPLC): 99.56%

While the foregoing provides a detailed description of the preferred embodiments of the invention, it is to be understood that the descriptions are illustrative only of the principles of the invention and not limiting. Furthermore, as many changes can be made to the invention without departing from the scope of the invention, it is intended that all material contained herein be interpreted as illustrative of the invention and not in a limiting sense.

We claim:

1. A process for preparing crystalline Form-SDI of Dasatinib (I), 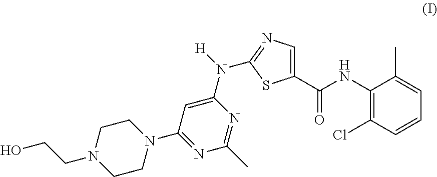 [SMILES: Cc1nc(Nc2ncc(C(=O)Nc3c(C)cccc3Cl)s2)cc(N2CCN(CCO)CC2)n1]
comprising the steps of:
a) Reacting N-(2-chloro-6-methylphenyl)-2-[6-chloro-2-methyl-4-pyrimidinyl) amino]-5-thiazole carboxamide (II) with 1-(3-Hydroxy)ethylpiperazine (III) in solvent 3-methylbutan-1-ol in absence of base; 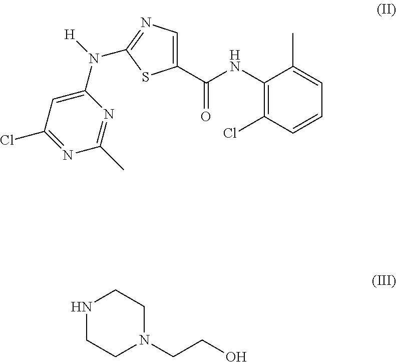 [SMILES: Cc1nc(Cl)cc(Nc2ncc(C(=O)Nc3c(C)cccc3Cl)s2)n1 ; OCCN1CCNCC1]
b) Raising the temperature of reaction mixture to a temperature above 100° C.;
c) Cooling the reaction mixture to room temperature;
d) Filtering the reaction mass and washing it with 3-methylbutan-1-ol;
e) Optionally, treating the mass obtained from step d) with X—CH₂—CH₂—CH₂—OH in presence of an organic base and 3-methylbutan-1-ol; wherein X is halogen;
f) Isolating the crystalline Form-SDI of Dasatinib (I).

2. A process for preparing crystalline Form-SDI of Dasatinib according to claim 1, wherein the organic base used in step e) is an amine or nitrogen-containing heterocyclic compound selected from diisopropyl ethyl amine, pyridine, methyl amine, trimethylamine or triethyl amine.

3. A process for preparing crystalline Form-SDI of Dasatinib according to claim 1, wherein step e) of treating the mass obtained from step d) with X—CH₂—CH₂—OH in presence of an organic base and 3-methylbutan-1-ol, further comprise the steps of—
i. Heating the reaction mixture to a temperature above 60° C.;
ii. Stirring of the reaction mixture for time ranging from 5-15 hrs;
iii. Cooling of the reaction mass to RT.

4. A process for preparing crystalline Form-SDI of Dasatinib according to claim 1, wherein the crystalline Form-SDI, isolated from step f) is further reacted to obtain pharmaceutically acceptable salts of Dasatinib selected from Dasatinib Glucuronate (A) or Dasatinib hydrochloride (B).

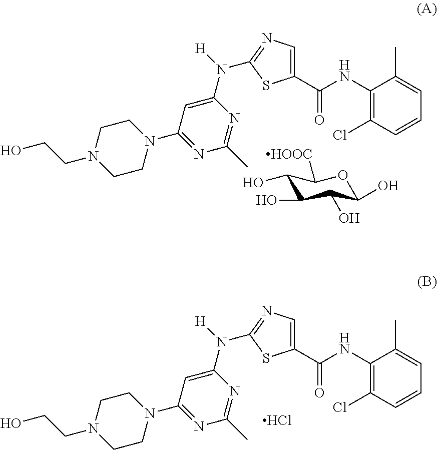

5. A process for preparing pharmaceutically acceptable salts of Dasatinib according to claim 4, wherein isolation of the pharmaceutically acceptable salt involves the treatment of the reaction mass with an organic solvent characterized by boiling point of less than 70° C.

6. Crystalline Form-SDI of Dasatinib, synthesized according to the process of claim-1, wherein the crystalline Form-SDI is characterized by X-ray powder diffraction pattern comprising of at least seven 2θ° peaks selected from the XRPD peak set of 5.8, 11.5, 12.7, 13.2, 17.3, 17.5, 18.1, 20.1, 20.5, 22.1, 25.4, 26.6, 26.8±0.20 2θ°.

7. Crystalline Form-SDI of Dasatinib, characterized by X-ray powder diffraction pattern comprising of at least seven 2θ° peaks selected from the XRPD peak set of 5.8, 11.5, 12.7, 13.2, 17.3, 17.5, 18.1, 20.1, 20.5, 22.1, 25.4, 26.6, 26.8±0.20 2θ°; IR spectrum having at least five absorption peaks selected from about 3390 cm⁻¹, 2923 cm⁻¹, 1621 cm⁻¹, 1615 cm⁻¹, 1537 cm⁻¹, 1316 cm⁻¹, 1061 cm⁻¹, 815 cm⁻¹ and 783 cm⁻¹; and DSC isotherm comprising at least two endothermic peaks ranging between—
a) 130° C. to 150° C.,
b) 160° C. to 175° C. or
c) 280° C. to 290° C.

8. Crystalline Form-SDI of Dasatinib according to claim-7, wherein the Crystalline Form-SDI is characterized by 3-methylbutan-1-ol content in range of 10-16% w/w.

9. Crystalline Form-SDI of Dasatinib according to claim 7, characterized by X-ray powder diffraction pattern, substantially according to FIG. 1 or, exhibiting a doublet diffraction angle peak at 23.6 and 23.8±0.20 2θ°, IR absorption spectrum substantially according to FIG. 2 and DSC isothermal pattern substantially according to FIG. 3.

10. A pharmaceutical composition comprising Crystalline Form-SDI of Dasatinib or its hydrate thereof, together with at least one or more pharmaceutically acceptable excipients.
